(19) United States
(12) Patent Application Publication
(10) Pub. No.: US 2006/0199221 A1
(43) Pub. Date: Sep. 7, 2006

(54) Correction for temperature dependence assays

(76) Inventor: Samad Talebpour, Richmond Hill (CA)

Correspondence Address: Ralph A. Dowell of DOWELL & DOWELL P.C., 2111 Eisenhower Ave, Suite 406, Alexandria, VA, 22314, US

(21) Appl. No.: 11/072,651

(22) Filed: Mar. 7, 2005

(51) Int. Cl. G01N 33/53; G06F 19/00

(52) U.S. Cl. 435/7.1; 702/19

(57) ABSTRACT

The present invention provides a method to calibrate an assay for variations or fluctuations in an assay parameter such as, but not limited to, temperature that affect the measured assay signal and thereby to calculate an analyte concentration with improved accuracy. The method is preferably performed in a manner such that the measured signal from a single additional standard per batch is sufficient to provide the required calibration.

### FIELD OF THE INVENTION

The present invention is related to assays and methods of correcting for variations in assays due to variations in external parameters such as temperature.

### BACKGROUND OF THE INVENTION

Assays of samples (i.e. biological, environmental etc) are routinely used to detect and measure the presence and the concentration of analytes such as drugs, pollutants, chemicals, contaminants, or the like. Regardless of the format for the assays, the analyte concentration is inferred from the dose-response curve. This can be done by finding the ordinate on the dose-response curve corresponding to the signal for the unknown concentration of analyte in the sample. The later quantity is given by the value of the abscissa. The dose-response curve is typically non-linear and it can be prepared by assaying standard samples containing known concentrations of the analyte. The number of standard samples and their concentrations are selected in order to determine the analyte concentrations with sufficient accuracy over the expected assay range.

Any variation in the reagents or assay conditions, however, can modify the actual relationship between the measured signal and analyte concentration from the initial dose-response curve. It is therefore necessary for an analyzer to control variables that affect the dose-response curve, such as temperature, with high precision. If the stabilization of these variables is imperfect, then it may be necessary to assay additional calibrators to correct for the deviation from the initial conditions (i.e. at the time when the initial dose-response curve was measured).

In order to obtain quantitatively accurate results, it is ideal to measure a dose-response curve via assays of known standards in parallel with assays for samples containing unknown concentrations of analyte (i.e. a simultaneous measurement of standards and unknowns). Unfortunately, this approach typically causes a significant reduction in assay throughput. This is particularly true for an assay with a nonlinear dose-response curve that requires many standards for accurate curve fitting, since each standard that is required must take the place of a sample. Not only does this limitation decrease the throughput of an analyzer, but it also adds significant cost and complexity. The onus on the user to store, handle and insert additional reagents (i.e. standards) is thus a major operational drawback of frequent calibration.

An alternative solution is to employ internal calibrators, for which the assay reagents are designed to give, in addition to a principal signal indicative of the analyte concentration, a calibration signal to correct for variations in the assay parameters. U.S. Pat. No. 5,648,274 (Chandler et al.) describes the use of a single internal calibrator in a comparative dual assay and U.S. Pat. No. 5,387,503 (Selmer et al.) describes the use of internal calibration by the addition of foreign analytes to samples and detection of both the target and foreign analytes at separate areas on a solid support. In a variation of these methods, the sequential addition to the assay reagents of a sample followed by the internal calibrator is described in U.S. Pat. No. 6,514,770 (Sorin). The disadvantage of using an internal calibrator is the requirement for dedicated reagents, which are not always commercially available, and an analyzer capable of detecting two signals. For these reasons, internal calibrators are not easily integrated into a commercial assay platform.

What is therefore required is an external calibration method that does not significantly impair assay throughput and also does not require the addition of separate calibrator reagents.

### SUMMARY OF THE INVENTION

The present invention overcomes the disadvantages of the prior art by providing a simple and accurate method for calibrating the dose-response curve of an assay against variations in assay parameters such as temperature or systematic variations in the accuracy of liquid handling. This is achieved by recognizing that the effect of these and other variations on the dose-response curve of assay is often a simple multiplicative scaling of the curve. The degree of scaling of the curve as a result of a variation in an assay parameter can therefore be accurately calibrated via a single calibration standard that is assayed with one or more unknown samples. By recording the change in the assay signal of the single calibration standard relative to its initial value at the time of measuring a complete dose-response curve (measured with a set of many standards at different concentrations), the dose-response curve can be scaled to accurately compensate for the effect of the variations.

Thus, in one aspect the present invention provides a method of calibrating a dose-response curve of an assay for a target analyte, where the effects of variations in the dose-response curve, due to fluctuations in an assay parameter, on the determination of an unknown quantity of analyte in a sample are reduced or eliminated, comprising the steps of:

a) performing a set of initial assays for a plurality of standards with known analyte concentrations and measuring an assay signal for each standard;

b) fitting said measured signals and said known analyte concentrations to a predetermined functional form for generating a first dose-response curve;

c) performing one or more assays, each for a sample with an unknown quantity of analyte, together with performing one or more assays for an additional standard with a known quantity of analyte;

d) calculating an average signal from said one or more assays of said additional standard and calculating a ratio of said average signal to a signal predicted by said first dose-response curve at the concentration of analyte in said additional standard;

e) multiplying said first dose-response curve by said ratio to obtain a calibrated dose-response curve; and

f) employing said calibrated dose-response curve to obtain one or more inferred concentrations of analyte from said one or more samples.

If the variations in an assay parameter produce correlated effects on standards with and without analyte, then the above method can be modified to provide a preferred embodiment of the invention. This correlation necessarily implies a finite and measurable assay signal for a standard lacking analyte. In such a case, it is possible to use a negative (i.e. zero analyte) standard for the calibration assay in the above method. However, the method may be further simplified by excluding the negative standard altogether and simply measuring a calibration assay performed in the absence of a standard, which may also serve as a “negative” calibrator for the purpose of calibrating the dose-response curve. Such a method advantageously does not require any additional reagents or calibration standards for the calibration of the dose-response curve.

Thus, in another aspect of the present invention, there is provided a method of calibrating a dose-response curve of an assay for a target analyte, where the effects of variations in the dose-response curve, due to fluctuations in an assay parameter, on the determination of an unknown quantity of an analyte in a sample are reduced or eliminated, comprising the steps of:

a) performing a set of initial assays for a plurality of standards with known analyte concentrations and measuring a signal for each assay;

c) fitting said measured signals and said known analyte concentrations to a predetermined functional form for generating a first dose-response curve;

d) performing one or more assays, each for a sample with an unknown quantity of analyte, together with performing one or more a additional assays without the addition of a standard and measuring a signal for each assay;

e) calculating an average signal from said one or more additional assays without the addition of a standard and calculating a ratio of said average signal to a signal predicted by said first dose-response curve at a concentration of analyte of zero

f) multiplying said first dose-response curve by said ratio to obtain a calibrated dose-response curve; and

g) employing said calibrated dose-response curve to obtain one or more inferred concentrations of said analyte from said one or more samples.

The preceding method requires, for its successful operation, that the dependence of the assay signal on variations in an assay parameter be correlated for standards with and without analyte. Unfortunately, this is not the case for several assays that produce a negligible or uncorrelated signal without the presence of analyte in a sample. This problem, however, can be circumvented by the addition of a small quantity of analyte to one of the reagents (or to the sample itself). The small quantity of analyte produces a finite, measurable and correlated signal even in the absence of analyte in the sample. Furthermore, if the addition of analyte is made to a reagent, then no additional calibrators or reagents are required for the calibration assay, as in the aforementioned method.

Thus, in a another aspect of the present invention, there is provided a method of calibrating a dose-response curve of an assay for a target analyte, where the effects of variations in the dose-response curve on the determination of an unknown quantity of an analyte in a sample are reduced or eliminated, comprising the steps of:

a) adding a known quantity of the analyte to one or more reagents, or to the sample itself, in said assay for use with all subsequent assays;

b) performing a set of initial assays for a plurality of standards with known analyte concentrations and measuring a signal for each assay;

c) fitting said measured signals and said known analyte concentrations to a predetermined functional form for generating a first dose-response curve,

d) performing one or more assays, each for a sample with an unknown quantity of analyte, together with performing one or more a additional assays without the addition of a standard and measuring a signal for each assay;

e) calculating an average signal from said one or more additional assays without the addition of a standard and calculating a ratio of the average signal to a signal predicted by said first dose-response curve at a concentration of analyte of zero;

f) multiplying said first dose-response curve by said ratio to obtain a calibrated dose-response curve; and

g) employing said calibrated dose-response curve to obtain one or more inferred concentrations of the analyte from said one or more samples wherein the step a) of adding a known quantity of the analyte to one or more reagents provides a correlation between variations in the signal from an assay for which no additional standard or sample is added and variations in a signal from an assay for a standard having a finite quantity of the analyte.

Although the above methods succeed in correcting for variations that cause multiplicative scaling of the dose-response curve, it does not address assays that exhibit both multiplicative scaling and translation of the dose-response curve. Assays which exhibit both types of multiplicative variations can, however, be corrected using a method in which two different standards with different known analyte concentrations are measured when performing assays for samples with unknown analyte concentrations. A measurement of two standards allows one to solve for the multiplicative scaling factor, which multiplies the entire dose-response curve, and the multiplicative translation factor, which multiplies the concentration variable in the dose response curve. The corrected dose-response curve can then be used to infer accurate analyte concentrations from unknown samples even when both types of multiplicative variations are present.

Thus, in a another aspect of the present invention, there is provided a method of calibrating a dose-response curve of an assay for a target analyte, where the effects of both multiplicative scaling and translation variations in the dose-response curve, due to fluctuations in an assay parameter, on the determination of an unknown quantity of analyte in a sample are reduced or eliminated, comprising the steps of:

a) performing a set of initial assays for a plurality of standards with known analyte concentrations and measuring an assay signal for each standard;

b) fitting said measured signals and said known analyte concentrations to a predetermined mathematical function for generating a first dose-response curve;

c) modifying said first dose-response curve with generated with said predetermined mathematical function by including a multiplicative scaling factor gₛ that multiplies all parameters in said predetermined mathematical function and a multiplicative translation factor gₜ that multiplies a concentration variable in said predetermined mathematical function, where said multiplicative scaling and translation parameters are unknown and represent possible variations in the first dose-response curve during future use of said assay, thereby obtaining a generalized dose-response curve;

d) performing one or more assays, each for a sample with an unknown quantity of analyte, together with performing assays for two additional standards, each standard having a different known concentration of analyte, where one or more assays is performed for each additional standard;

e) calculating an average signal from said assays of each of said additional standards, thereby obtaining two data pairs, each comprising a known concentration and a measured assay signal;

f) generating two equations using said two data pairs and said generalized dose-response curve and solving said two equations for values of said multiplicative scaling factor and said multiplicative translation factor;

g) generating a new dose-response curve by inserting said values of said multiplicative scaling factor and said multiplicative translation factor into said generalized dose-response curve; and

h) employing said new dose-response curve to obtain one or more inferred concentrations of said analyte from said one or more samples.

A further understanding of the functional and advantageous aspects of the invention can be realized by reference to the following detailed description and drawings.

### BRIEF DESCRIPTION OF THE DRAWINGS

The invention will be more fully understood from the following detailed description thereof taken in connection with the accompanying drawings, which form a part of this application, and in which:

FIG. 1 illustrates the dose-response curve of a fictitious assay before (solid line) and after (dotted line) the addition of a small quantity of analyte to the reagents.

FIG. 2 plots the rate of change in absorbance after an incubation time of 20 minutes for an EDDP (a metabolite of methadone) assay at two different temperatures. The solid line shows the dose-response fitted to the data measured at T=27° C.

FIG. 3 plots the dependence of the rate of change of absorbance for an EDDP assay on temperature, at several different analyte concentrations.

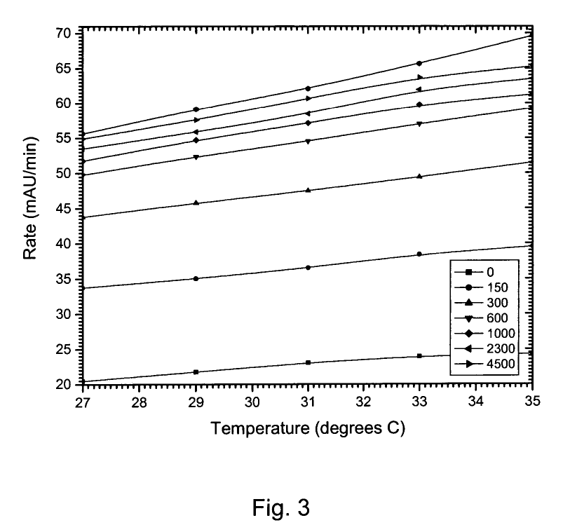

FIG. 4 shows the measured rate of change of absorbance of an assay for EDDP (a metabolite of methadone) at two different temperatures, where a small amount of analyte has been added to one of the reagents in order to obtain a nonzero signal for zero analyte. The solid line shows the dose-response fitted to the data measured at T=27° C., and the dotted line shows the dose-response curve calibrated to T=35° C. according to the method of the invention.

FIG. 5 illustrates the chemical reaction that occurs to provide the assay signal in an assay for creatinine.

FIG. 6 shows the measured rate of change of absorbance for a creatinine assay for two different temperatures. The solid line shows the dose-response fitted to the data measured at T=27° C., and the dotted line shows the dose-response curve calibrated to T=35° C., illustrating the failure of the method when there is negligible signal in the absence of analyte.

FIG. 7 plots the dependence of the rate of change of absorbance for a creatinine assay on temperature, at several different analyte concentrations, showing the lack of correlation of temperature dependence between signals with and without analyte.

FIG. 8 shows the temperature dependence of the creatinine assay signal in the absence of analyte.

FIG. 9 shows the measured rate of change of absorbance for a creatinine assay for two different temperatures, where a small amount of analyte has been added to one of the reagents in order to obtain a nonzero signal for zero analyte. The solid line shows the dose-response fitted to the data measured at T=27° C., and the dotted line shows the dose-response curve calibrated to T=35° C. according to the method of the invention.

FIG. 10 shows the measured rate of change of absorbance for an ethanol assay for two different temperatures, where a small amount of analyte has been added to one of the reagents in order to obtain a nonzero signal for zero analyte. The solid line shows the dose-response fitted to the data measured at T=27° C., and the dotted line shows the dose-response curve calibrated to T=35° C. according to the method of the invention.

FIG. 11 shows the measured rate of change of absorbance of an assay for BGZ (a metabolite of cocaine) at two different temperatures. The solid line shows the dose-response fitted to the data measured at T=28° C., and the dotted line shows the dose-response curve calibrated to T=35° C. using both multiplicative scaling and translation factors. The dashed line illustrates the poor correction obtained by using a multiplicative scaling factor alone.

### DETAILED DESCRIPTION OF THE INVENTION

The present invention improves upon the prior art methods described above by providing a simple and effective method for correcting a measured dose-response curve for variations in assay parameters. This is achieved by making use of the fact that several types of external variations, such as the effects of temperature and systematic changes in the volumes of dispensed or aspirated reagents, affect the dose-response curve in a global fashion. Indeed, it has been experimentally found that many of such variations produce a dose-response curve that is a simple multiplicative scaling of the original dose-response curve measured with multiple standards.

Furthermore, it has been found that in most cases, the variations in the dose-response curve are monotonic with respect to the variation, which avoids potential erroneous results from double-valued functions.

The present invention will be illustrated using the case where the assay parameter is temperature and the variation in the dose-response curve is due to fluctuations in temperature.

In a simple mathematical example, an assay with the following dose-response curve measured at temperature T₀ is considered:
S(C,T)|T₀=f(C), (1)
where C is the analyte concentration and S(C,T) is the assay signal. In general, the response at an arbitrary temperature is given by
S(C,T)=f(C)g(C,T−T₀). (2)

In many cases, the general function g(C,T−T₀) in equation (2) can be approximated over a certain restricted region of parameter space as being independent of analyte concentration, in which case the function S(C, T) is separable and
g(C,T−T₀)≈g(T−T₀). (3)
One readily observes that the effect of temperature on the dose-response curve is a simple scaling of the curve. In this case, the new dose-response curve can be calibrated via the measurement of a single standard with a concentration C′, which enables the measurement of g(T−T₀):

$$\begin{matrix}\frac{S ( C , T )}{S ( C , {T}_{0} )} = \frac{S ( {C}^{′} , T )}{S ( {C}^{′} , {T}_{0} )} = g ( T - {T}_{0} ) , \\ giving & ( 4 ) \\ S ( C , T ) = S ( C , {T}_{0} ) [ \frac{S ( {C}^{′} , T )}{S ( {C}^{′} , {T}_{0} )} ] . & ( 5 )\end{matrix}$$

It is important to note that the above calibration scheme does not require the knowledge of either T₀ or the functional nature of g(T−T₀), which can take any form.

In this embodiment, a method of calibrating a dose-response curve of an assay for a target analyte, where the effects of variations in the dose-response curve, due to fluctuations in an assay parameter, on the determination of an unknown quantity of analyte in a sample are reduced or eliminated, comprises the steps of performing a set of initial assays for a plurality of standards with known concentration of an analyte and measuring an assay signal for each standard; fitting the measured signals and known analyte concentrations to a predetermined functional form for generating a first dose-response curve; performing one or more assays, each for a sample with an unknown quantity of analyte, together with performing one or more assays for an additional standard with a known quantity of analyte; calculating the ratio of the average signal from the one or more assays of the additional standard to the signal predicted by the first dose-response curve at the concentration of analyte in the additional standard; multiplying the first dose-response curve by the ratio to obtain a calibrated dose-response curve; and employing the calibrated dose-response curve to obtain one or more inferred concentrations of analyte from the one or more samples.

In a preferred embodiment of the above invention, the assay calibration is achieved without the need for either a standard or a calibration reagent. This is achieved by modifying the method described in the preceding paragraph so that the assays for an additional standard are replaced by assays without the addition of a standard. This assay serves as a negative calibration and advantageously does not require the purchase, storage or handling of any additional calibration reagents. In this reagentless calibration method, the initial dose-response curve is modified according to the equation

$$S ( C , T ) = S ( C , {T}_{0} ) [ \frac{S ( 0 , T )}{S ( 0 , {T}_{0} )} ] . \qquad ( 6 )$$

The above method, however, fails to provide a suitable calibration when the signal produced in the absence of sample is not large enough to provide a sufficient signal-to-noise ratio. The method also fails when the temperature dependence of the signal produced in the absence of analyte is not correlated with the temperature dependence of the assay signal produced by a reaction involving actual analyte. This may arise in assays where the analyte plays a critical role in the development of an assay signal, unlike other assays where the signal lies on top of a background, as in enzyme assays for example. In such assays with either a negligible or uncorrelated negative signal, one can simply use the method of calibration shown in equation 5, where a calibrator reagent with a nonzero analyte concentration is used. The use of a nonzero calibrator reagent, however, removes the advantages of a reagentless calibration scheme and leads to a less practical method.

Another method of calibrating an assay with a negligible signal in the absence of analyte is to add a small quantity of analyte to one of the reagents, or alternatively to the sample itself. This method causes a nonzero analyte concentration to be present even in the absence of analyte in the sample. An assay signal with a suitable signal-to-noise is therefore obtained regardless of the analyte concentration, enabling the use of equation 6 to provide a reagentless calibration scheme.

In this embodiment of the method of calibrating a dose-response curve of an assay for a target analyte, where the effects of variations in the dose-response curve on the determination of an unknown quantity of an analyte in a sample are reduced or eliminated, comprises the steps of adding a known quantity of the analyte to one or more reagents for use with all subsequent assays, performing a set of initial assays for a plurality of standards with known concentration of the analyte and measuring a signal for each assay; fitting said measured signals and said known analyte concentrations to a predetermined functional form for generating a first dose-response curve, performing one or more assays, each for a sample with an unknown quantity of analyte, together with performing one or more additional assays without the addition of a standard and measuring a signal for each assay, calculating an average signal from said one or more additional assays without the addition of a standard and calculating a ratio of the average signal to a signal predicted by said first dose-response curve at a concentration of analyte of zero; multiplying the first dose-response curve by the ratio to obtain a calibrated dose-response curve, and employing the calibrated dose-response curve to obtain one or more inferred concentrations of the analyte from the one or more samples.

The step of adding a known quantity of the analyte to one or more reagents provides a correlation between variations in the signal from an assay for which no additional standard or sample is added and variations in a signal from an assay for a standard having a finite quantity of the analyte.

Alternatively, instead of the first step of adding a known quantity of the analyte to one or more reagents used in the assay, a known quantity of the analyte could instead be added just to the sample.

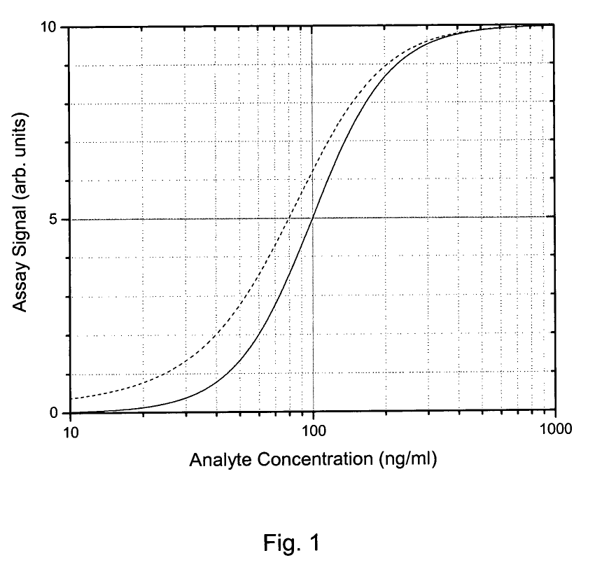

Although the addition of a known quantity of analyte to one of the reagents modifies all subsequent measurements of samples, this modification is built into the measured dose-response curve and poses no problem. This is illustrated in FIG. 1, where the dose-response curve of a fictitious assay with zero signal in the absence of analyte is shown by the solid line. This curve is generated by the logistic function

$$S ( C , {T}_{0} ) = \frac{10}{1 + {( \frac{100}{C} )}^{2.71}} , \qquad ( 7 )$$

where C is given in units of ng/ml. The addition of a quantity of analyte to one of the reagents, corresponding to a sample concentration of 20 ng/ml, causes the dose-response curve to shift upwards, as shown by the dashed line. This new curve represents the dose-response curve of the inventive assay, whereby a non-zero assay signal is obtained even in the absence of sample analyte. The functional form of the new curve is given by

$$S ( C , {T}_{0} ) = \frac{10}{1 + {( \frac{100}{C + 20} )}^{2.71}} , \qquad ( 8 )$$

which is a modified form of logistic function. This functional form is used to determine the fitting parameters when measuring the initial set of standards for the generation of the dose-response curve.

As can be seen in FIG. 1, the addition of a small amount of analyte to the reagents produces a significant and measurable signal even in the absence of analyte in the sample. It is important to note that although this signal is small relative to the signals obtained for higher analyte concentrations, the signal-to-noise ratio of this signal is typically similar or equal to that of the higher signal. This is because of the fact that the typical sources of error in such an assay are due to accuracy in liquid handling and signal measurement that affect all readings equally. Interestingly, the slope of the dose-response curve in the low-concentration region is not compromised by the addition of sample. This is even true for concentrations below that of the corresponding sample analyte concentration of the analyte added to the reagents.

Although many types of assays produce dose-response curves that exhibit simple multiplicative scaling with variations in assay parameters, some assays exhibit a more complex sensitivity that is described by a combination of multiplicative scaling and multiplicative translation. In such cases, the methods described above will fail to correct for the translation component of the variation in the dose-response curve, ultimately causing errors in the inferred analyte concentration.

A final embodiment of the invention provides a method for correcting for such dual variations in the dose-response curve. The multiplicative scaling and multiplicative translation factors, which are henceforth referred to by gₛ and gₜ, represent two unknowns that must be obtained to correct the dose-response curve. These unknowns can be determined by measuring the signal from two standards with different analyte concentrations.

The correction of a sigmoidal dose-response curve for multiplicative scaling and translation variations is now considered. A sigmoidal dose-response curve is generally represented by the mathematical function

$$S ( C ) = {a}_{2} + \frac{{a}_{1} - {a}_{2}}{1 + {( \frac{C}{{a}_{3}} )}^{a 4}} , \qquad ( 9 )$$

where S is the assay signal, C is the analyte concentration, and a₁-a₄ are parameters. A variation in an assay parameter that produces a multiplicative scaling and translation causes the dose-response curve to take the new form

$$S ( C ) = {g}_{s} [ {a}_{2} + \frac{{a}_{1} - {a}_{2}}{1 + {( \frac{{g}_{t} C}{{a}_{3}} )}^{a 4}} ] . \qquad ( 10 )$$

The new dose-response curve is obtained as follows. Two standards with concentrations C₁ and C₂ are assayed and their respective signals S₁ and S₂ are recorded. The two data points S₁(C₁) and S₂(C₂) and equation (10) are then used to construct two equations with the two unknowns gₛ and gₜ. The solution of these equations provides gₛ and gₜ, enabling the accurate mathematical construction of the new dose-response curve shown in equation (10).

A method for correcting for multiplicative scaling and translation of a dose-response curve is therefore provided as follows. A first dose-response curve is generated by measuring the signals from several standards with known analyte concentrations and fitting the data to a known mathematical function. A new dose-response curve describing the assay following variations causing multiplicative scaling and translation is obtained by multiplying the entire initial dose-response equation by a scaling factor and multiplying the concentration variable in the initial dose-response equation by a translation parameter. When assaying one or more samples with unknown analyte concentrations at a later time when variations may have occurred, two additional standards with known different concentrations are assayed as well. The new dose-response curve that is valid for the conditions under which the unknown samples are assayed is obtained by solving two equations for the dose-response curve using two known signals and analyte concentrations from the assayed additional standards for the unknown scaling and translation factors. The new dose-response curve is then used to infer the concentrations of analyte in the samples that were assayed with the two additional standards.

These embodiments of the aforementioned invention are henceforth discussed with reference to several non-limiting examples.

### EXAMPLE 1

### Homogeneous Competitive Enzyme Immunoassay for EDDP

In this example, a homogeneous competitive enzyme immunoassay (EIA) is performed for EDDP, a primary metabolite of methadone. The assay involves the competition between analyte in a sample and an enzyme-labeled conjugate (i.e. analyte labeled with an enzyme) for a limited number of antibody binding sites in a homogeneous reagent. The enzyme acts upon chromogenic substrates to generate a reaction product with a change in optical absorbance within a narrow spectral range, thereby producing the assay signal. The rate of product generation depends on the activity of the enzyme, which is modified upon the binding of the enzyme-labeled conjugate to an antibody. The enzyme activity, which is therefore related to the concentration of bound analyte, is determined by a photometric measurement.

This assay has two reagents: (1) R₁, which includes monoclonal antibodies against EDDP, and the substrates for the enzyme (glucose-6-phosphate, G6P and nicotinamide adenine dinucleotide, NAD), and (2) R₂, which includes the enzyme-labelled conjugate, i.e. glucose-6-phosphate dehydrogenase, G6PDH, labeled with EDDP.

An experiment was conducted in which the dose-response curve was generated using seven standards with analyte concentrations in the range 0-4500 ng/ml. Using a microtiter plate, 80 μL of R₁ and 30 μL of R₂ were mixed with 20 μL of each standard separately in a row of wells. The contents of the microtiter plate were vortexed for 30 seconds and then the absorbance at 340 nm, OD(t), was measured at intervals of 30 seconds for a total duration of 30 minutes. The plate was maintained at a constant temperature during each measurement.

The rate of change in absorbance was calculated following an elapsed time of 20 minutes after addition of the reagents, i.e.

$$\frac{ⅆ OD ( t )}{ⅆ t} {\vert}_{t = 20} .$$

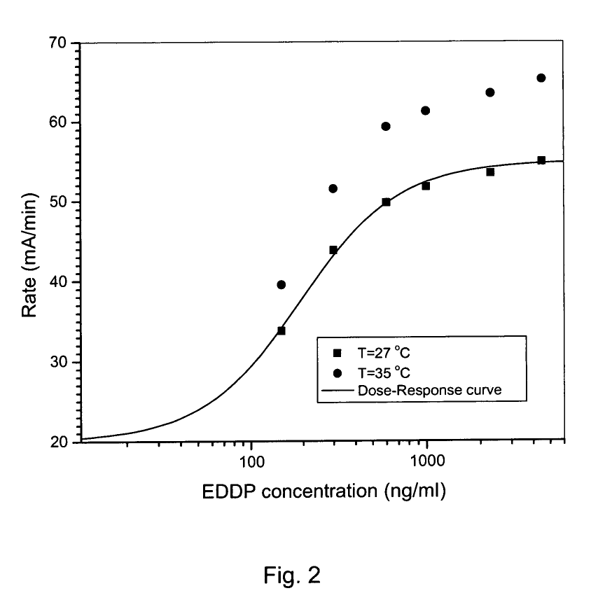

These values are plotted in FIG. 2 for two different temperatures of the microtiter plate. A sigmoidal curve was fitted to the measured points at T=27° C. to obtain the dose-response curve shown in FIG. 1. As is clearly seen in the figure, it is not possible to use the dose-response curve measured at 27° C. to accurately determine the analyte concentrations corresponding to measurements at 35° C.

The dose-response curve shown in FIG. 1 indicates that the assay has a non-zero signal with a high signal-to-noise ratio in the absence of analyte in the sample. This indicates that a preferred method of the present invention, in which a reagentless calibration scheme is employed, can be used to calibrate the assay. This is further validated by the data shown in FIG. 2, where the correlation between the temperature dependence of the assay signal at various analyte concentrations (including zero analyte) is clearly evident.

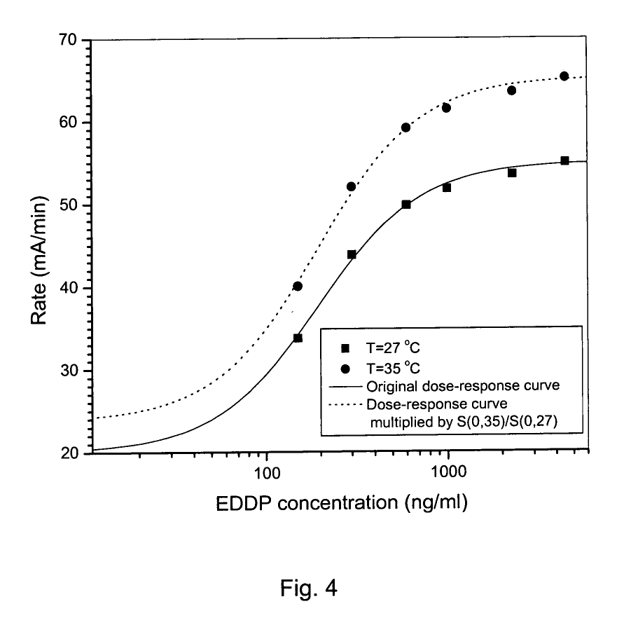

The dose-response curve was calibrated for 35° C. according to the prescription given in equation 5, with T₀=27° C. and T=35° C. The resulting dose-response curve, corrected for the temperature variation is shown along with the original dose-response curve in FIG. 4. The calibrated dose response curve accurately fits with the data measured at 35° C., validating the reagentless calibration scheme of the present invention.

### EXAMPLE 2

### Homogeneous Assay for Creatinine

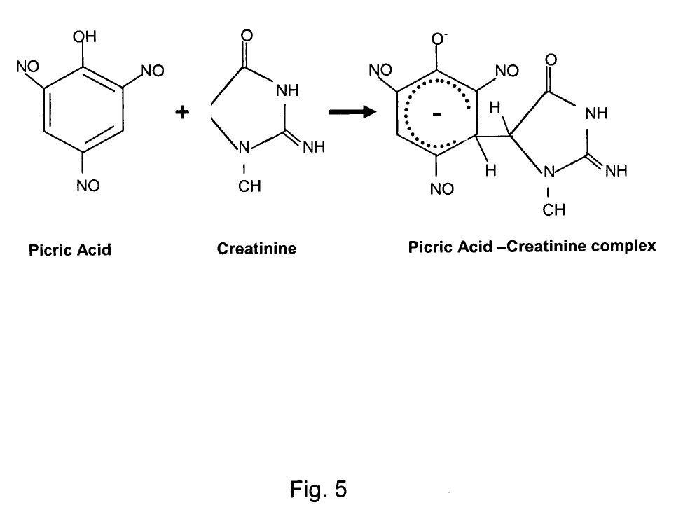

The application of the invention is also considered in a second example involving a commercially available assay for creatinine. This assay involves two reagents: (1) R₁ containing, principally, a high pH buffer, and (2) R₂ containing picric acid. The reaction between picric acid and creatinine is pictorially shown in FIG. 5.

The formation of a complex between creatinine and picric acid shifts the edge of the absorption spectrum in the visible region to higher wavelengths. The extent of the shift is related to the magnitude of the absorbance signal, OD(t), at a fixed wavelength chosen within the range of 505 nm to 560 nm. This absorbance measurement is made at a wavelength located on the red tail of the absorbance peak for picric acid and an increase in the magnitude of the absorbance signal can be directly related to the displacement of the peak to longer wavelengths. The rate of change of absorbance,

$$\frac{ⅆ OD ( t )}{ⅆ t} {\vert}_{t = {t}_{f}} ,$$

or alternatively the difference between the initial and final absorbance values OD(t_{f})−OD(t₀), can be chosen to provide a quantitative response for the assay, where t₀ and t_{f} (t_{f}>t₀) represent fixed periods of elapsed time measured after the sample and reagents are mixed together. In the specific experiment that will be described in this example, the absorbance difference was measured, i.e. with S(C,T)≡OD(t=20 min.)−OD(t=0), where C is the concentration of creatinine in sample and T is the incubation temperature.

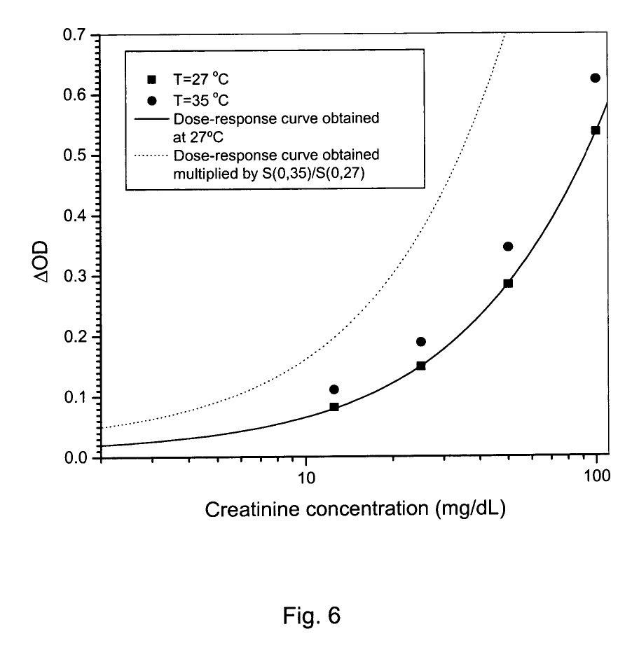

The dose-response curve was generated using five standards with concentrations of creatinine in the range 0-100 mg/dL. The assay was also performed in a microtiter plate where 100 μL of R₁ and 30 μL of R₂ were mixed with 10 μL of each standard in a row of wells. The contents of the microtiter plate were initially vortexed for 30 seconds and then the absorbance at a wavelength of 535 nm was measured immediately, giving the initial value OD(0). Following an incubation time of 20 minutes, the final absorbance OD(20 min.) was measured. The change in absorbance between these measurements is plotted in FIG. 6 for two different temperatures of the microtiter plate. A logistic curve, fitted to the data measured at T=27° C., was used to generate the dose-response curve shown in FIG. 6. Clearly the dose-response curve measured at 27° C. could not be used for an accurate determination of analyte concentrations corresponding to a temperature of 35° C.

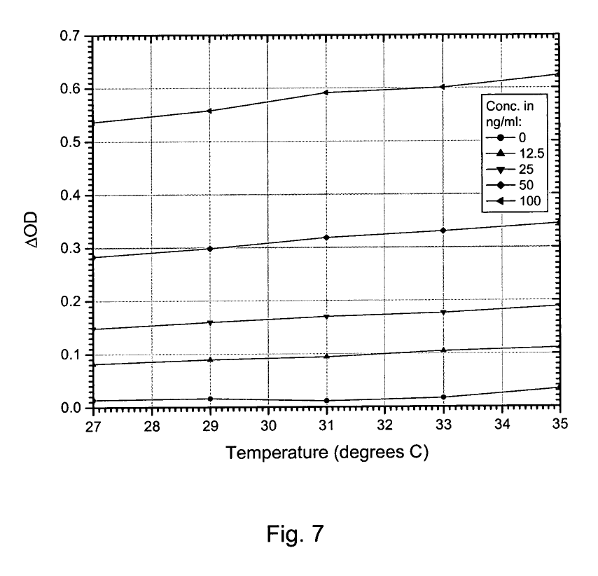

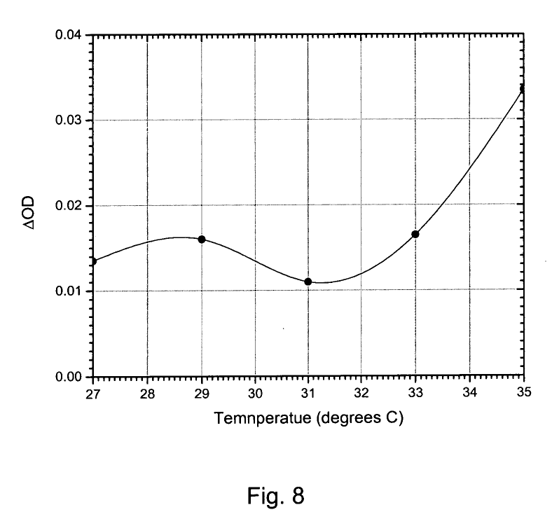

It is also clear from FIG. 6 that the calibration scheme according to equation 5 also produces an erroneous result (shown by the dotted line). The reason for this failure, as can be seen in FIG. 7, is the negligible and uncorrelated assay signal that is obtained in when no analyte is present in the sample. Although the curves shown in FIG. 7 for different finite analyte concentrations have a correlated temperature dependence, the signal obtained without analyte is small and noisy. This can be seen in more detail in FIG. 8, where the temperature dependence of the assay signal with no analyte is shown. The variations observed in this figure are in fact within the experimental error of the absorbance reader and thus do not reflect any real phenomena.

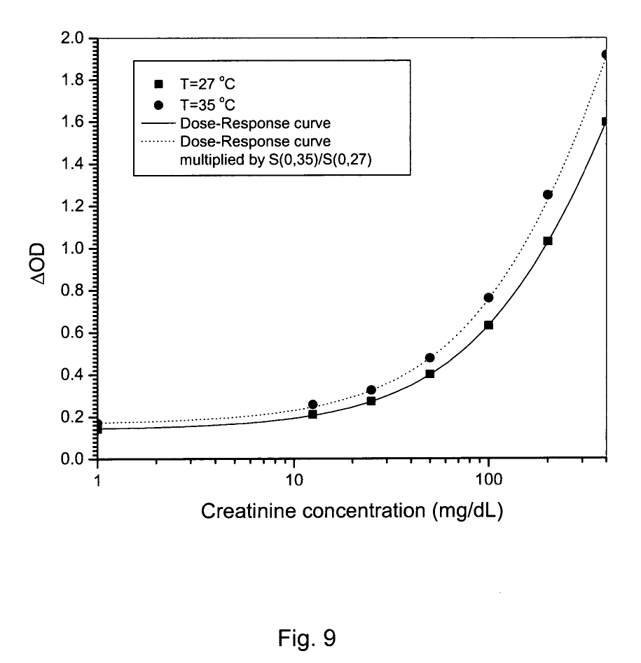

This problem was surmounted using the method of the invention described above, whereby a small amount of analyte is added to the reagents in order to produce a measurable signal that shares a correlated temperature dependence with signals produced by samples with finite quantities of analyte. In this example, 10 μL of a standard containing 25 mg/dL of creatinine was added to 90 μL of the original reagent R₁ and the assay was performed at two different temperatures, 27° C. and 35° C. In a microtiter plate, the 100 μL aliquots of the new reagent R₁ were mixed with 30 μL of the original reagent R₂ and 10 μL of each standard in separate wells. As before, the contents of the microtiter plate were initially vortexed for 30 seconds and then the absorbance at 535 nm was measured immediately, giving the initial value OD(0). Following an incubation time of 20 minutes, the final absorbance OD(20 min.) was measured. The results are illustrated in FIG. 9 for the two different temperatures of the plate. A logistic curve was fitted to the measured points at T=27° C. to obtain the dose-response curve shown in FIG. 9. Again, it is not possible to use the dose-response curve measured at 25° C. to accurately determine the analyte concentrations corresponding to measurements at T=35° C.

However, as described above, the addition of analyte to R₁ enables the use of equation 5 for the calibration of the dose-response curve. The resulting dose-response curve, calibrated for use at 35° C., is shown by the dotted curve in FIG. 9. The calibrated dose response curve again accurately fits with the data measured at 35° C., validating the modified reagentless calibration method.

### EXAMPLE 3

### Ethanol Assay

The third example considered is a commercially available ethanol assay. The assay has two reagents; R₁, which contains a Tris-based buffer, and R₂, which contains an enzyme, alcohol dehydrogenase (ADH), and a substrate, nicotinamide adenine dinucleotide (NAD). The enzyme-catalysed reaction is described below:

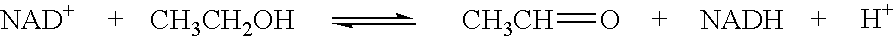

As the concentration of the product NADH increases, there is a corresponding increase in the measured absorption at 340 nm. A suitable procedure is to measure the absorbance, OD(t), at two fixed times, t_{f} and t₀, and the assay signal can be related to the magnitude of the difference, S(C,T) ≡OD(t_{f})−OD(t₀).

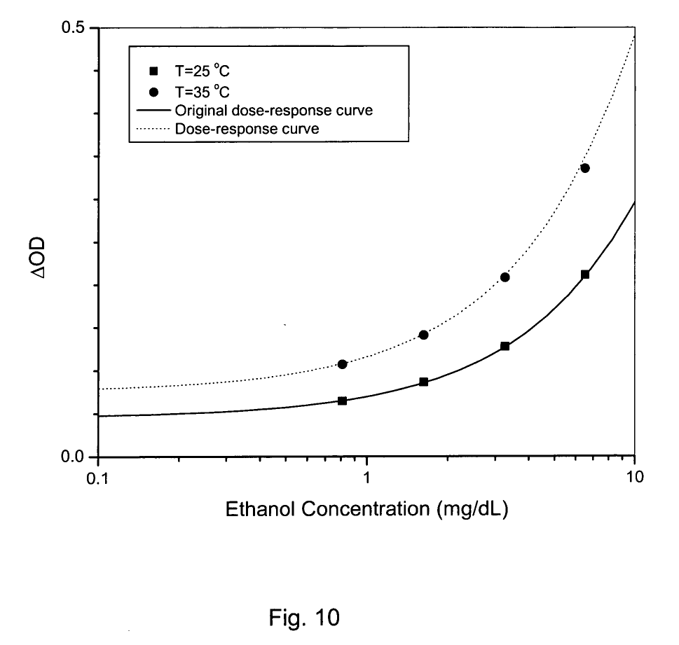

As with the example of the creatinine assay, the signal S(C,T) is not correlated with S(0,T) and it is necessary to modify the reagents to achieve a correlation. For this purpose, 2.25 mg of pure ethanol was added per 80 μL of the original reagent R₁. The dose-response curve was generated using six standards with concentrations of ethanol in the range 0-15 mg/dL. The assay was performed in a microtiter plate where 80 μL of the new reagent R₁ and 25 μL of R₂ were mixed with 8 μL of each standard in a row of wells. The microtiter plate was initially vortexed for 30 seconds and then the absorbance at 340 nm, OD(t), was measured at intervals of 0.5 minute. The initial absorbance, measured immediately after the mixture was vortexed, i.e OD(0), was subtracted from the absorbance measured after an elapsed time of 20 minutes, OD(20). This data is illustrated in FIG. 10 as a function of the concentration of ethanol. The initial dose-response curve was obtained by fitting a logistic curve to the measured signal at T=25° C.

With the addition of ethanol to R₁, the initial dose-response curve exhibits a large signal for zero analyte that is correlated to signals produced in the presence of analyte. This enables the use of equation 5 to generate a dose-response curve that is calibrated for T=35° C., as shown by the dotted line in FIG. 10. This calibrated dose-response curve lies on top of the measured data at 35° C., once again confirming the utility of the reagentless calibration method.

### EXAMPLE 4

### Homogeneous Competitive Enzyme Immunoassay for BZG

In this example, a homogeneous competitive enzyme immunoassay (EIA) is performed for BZG, a metabolite of cocaine. The assay involves the competition between analyte in a sample and an enzyme-labeled conjugate (i.e. analyte labeled with an enzyme) for a limited number of antibody binding sites in a homogeneous reagent, as in the EDDP assay described in Example 1.

This assay also has two reagents: (1) R₁, which includes monoclonal antibodies against BZG, and the substrates for the enzyme (glucose-6-phosphate, G6P and nicotinamide adenine dinucleotide, NAD), and (2) R₂, which includes the enzyme-labelled conjugate, i.e. glucose-6-phosphate dehydrogenase, G6PDH, labeled with BZG.

An experiment was conducted in which the dose-response curve was generated using seven standards with analyte concentrations in the range 0-3000 ng/ml. Using a microtiter plate, 80 μL of R₁ and 30 μL of R₂ were mixed with 20 μL of each standard separately in a row of wells. The contents of the microtiter plate were vortexed for 30 seconds and then the absorbance at 340 nm, OD(t), was measured at intervals of 10 seconds for a total duration of 3 minutes. The plate was maintained at a constant temperature during each measurement. The rate of change in absorbance was calculated following an elapsed time of 3 minutes after addition of the reagents, i.e.

$$\frac{ⅆ OD ( t )}{ⅆ t} {\vert}_{t = 3} .$$

A sigmoidal curve was fitted to the measured points to obtain a dose-response curve.

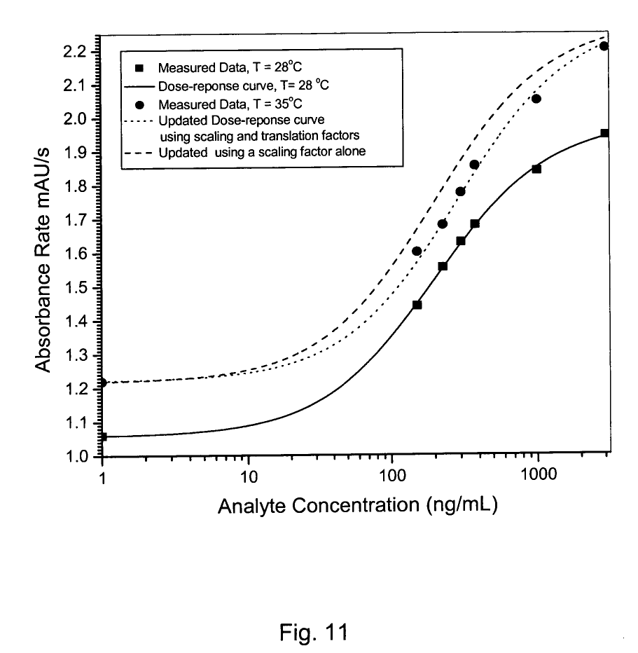

The above procedure was first performed at a constant temperature of 28° C., and the resulting measured data points and fitted dose-response curve are shown in FIG. 11. The same assay procedure was again performed at a temperature of 35° C., with the resulting data points also shown in FIG. 11. As in the case of the EDDP assay of Example 1, the data points measured at 35° C. do not lie on the dose-response curve obtained at 28° C. However, in this case, an attempt to modify the initial dose-response curve by multiplicative scaling of the signals obtained at a concentration of zero (shown by the dashed curve) failed to produce a new dose-response curve that agreed with the data measured at higher concentrations.

The failure of multiplicative scaling alone to correct for the variation in the dose-response curve indicates that this assay also requires a correction based on multiplicative translation. The aforementioned method of correcting for both multiplicative scaling and translation of the dose-response curve was therefore used to provide the necessary correction. The signals from the two standards at concentrations of 0 and 300 ng/ml were used to solve for the scaling and translation factors of the modified sigmoidal function. The new dose-response curve with the correct correction factors is shown in FIG. 11 by the dotted line. This new dose-response curve accurately fits will all data points across the entire concentration range of the assay, validating the method.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

Therefore what is claimed is:

1. A method of calibrating a dose-response curve of an assay for a target analyte, where the effects of variations in the dose-response curve, due to fluctuations in an assay parameter, on the determination of an unknown quantity of analyte in a sample are reduced or eliminated, comprising the steps of:
a) performing a set of initial assays for a plurality of standards with known analyte concentrations and measuring an assay signal for each standard;
b) fitting said measured signals and said known analyte concentrations to a predetermined functional form for generating a first dose-response curve;
c) performing assays for one or more samples with unknown quantities of analyte, together with performing one or more assays for an additional standard with a known quantity of analyte;
d) calculating an average signal from said one or more assays of said additional standard and calculating a ratio of said average signal to a signal predicted by said first dose-response curve at the concentration of analyte in said additional standard;
e) multiplying said first dose-response curve by said ratio to obtain a calibrated dose-response curve; and
f) employing said calibrated dose-response curve to obtain one or more inferred concentrations of said analyte from said assays for one or more samples.

2. The method according to claim 1 including repeating steps c) to f) for subsequent assays for one or more samples with unknown quantities of analyte.

3. The method according to claim 1 wherein the variation in the dose-response curve is caused by fluctuations or changes in temperature.

4. The method according to claim 1 wherein the variation in the dose-response curve is caused by fluctuations or changes in a quantity of aspirated reagents used in said assays.

5. The method according to claim 1 wherein the variation the dose-response curve is caused by fluctuations or changes in a quantity of dispensed reagents used in said assays.

6. The method according to claim 1 wherein the concentration of analyte in said additional standard is zero and where a correlation exists between variations in a signal from an assay for a standard in which there is no analyte and variations in a signal from an assay for a standard having a finite quantity of analyte.

7. A method of calibrating a dose-response curve of an assay for a target analyte, where the effects of variations in the dose-response curve, due to fluctuations in an assay parameter, on the determination of an unknown quantity of analyte in a sample are reduced or eliminated, and wherein a correlation exists between variations in a signal from an assay for which no additional standard or sample is added and variations in a signal from an assay for a standard having a finite quantity of analyte, comprising the steps of:
a) performing a set of initial assays for a plurality of standards with known concentration of an analyte and measuring a signal for each assay;
b) fitting said measured signals and said known analyte concentrations to a predetermined functional form for the generation of a first dose-response curve,
c) c) performing assays for one or more samples with unknown quantities of analyte, together with performing one or more additional assays without the addition of a standard, and measuring a signal for each assay;
d) calculating an average signal from said one or more additional assays without the addition of a standard and calculating a ratio of said average signal to a signal predicted by said first dose-response curve at a concentration of analyte of zero;
e) multiplying said first dose-response curve by said ratio to obtain a calibrated dose-response curve; and
f) employing said calibrated dose-response curve to obtain one or more inferred concentrations of said analyte from said one or more samples.

8. The method according to claim 7 including repeating steps c) to f) for subsequent assays for one or more samples with unknown quantities of analyte.

9. The method according to claim 7 wherein the variation in the dose-response curve is caused by fluctuations or changes in temperature.

10. The method according to claim 7 wherein the variation in the dose-response curve is caused by fluctuations or changes in a quantity of aspirated reagents used in said assays.

11. The method according to claim 7 wherein the variation the dose-response curve is caused by fluctuations or changes in a quantity of dispensed reagents used in said assays.

12. A method of calibrating a dose-response curve of an assay for a target analyte, where the effects of variations in the dose-response curve, due to fluctuations in an assay parameter, on the determination of an unknown quantity of an analyte in a sample are reduced or eliminated, comprising the steps of:
a) adding a known quantity of the analyte to one or more reagents for use with all subsequent assays;
b) performing a set of initial assays for a plurality of standards with known analyte concentrations and measuring a signal for each assay;
c) fitting said measured signals and said known analyte concentrations to a predetermined functional form for generating a first dose-response curve;
d) performing assays for one or more samples with unknown quantities of analyte, together with performing one or more additional assays without the addition of a standard and measuring a signal for each assay;
e) calculating an average signal from said one or more additional assays without the addition of a standard and calculating a ratio of the average signal to a signal predicted by said first dose-response curve at a concentration of analyte of zero;
f) multiplying said first dose-response curve by said ratio to obtain a calibrated dose-response curve; and
g) employing said calibrated dose-response curve to obtain one or more inferred concentrations of the analyte from said one or more samples wherein the step a) of adding a known quantity of the analyte to one or more reagents provides a correlation between variations in the signal from an assay for which no additional standard or sample is added and variations in a signal from an assay for a standard having a finite quantity of the analyte.

13. The method according to claim 12 including repeating steps d) to g) for subsequent assays for one or more samples with unknown quantities of analyte.

14. The method according to claim 13 wherein the first additional assay represents a standard with no analyte.

15. The method according to claim 12 wherein the variation in the dose-response curve is caused by fluctuations or changes in temperature.

16. The method according to claim 12 wherein the variation in the dose-response curve is caused by fluctuations or changes in a quantity of aspirated reagents used in said assays.

17. The method according to claim 12 wherein the variation the dose-response curve is caused by fluctuations or changes in a quantity of dispensed reagents used in said assays.

18. The method according to claim 12 wherein said small quantity of analyte is added to a reagent that does not react with said analyte.

19. A method of calibrating a dose-response curve of an assay for a target analyte, where the effects of variations in the dose-response curve, due to fluctuations in an assay parameter, on the determination of an unknown quantity of an analyte in a sample are reduced or eliminated, comprising the steps of:
a) adding a known quantity of analyte to the sample, in said assay for use with all subsequent assays;
b) performing a set of initial assays for a plurality of standards with known analyte concentrations and measuring a signal for each assay;
c) fitting said measured signals and said known analyte concentrations to a predetermined functional form for generating a first dose-response curve,
d) performing assays for one or more samples with unknown quantities of analyte, together with performing one or more additional assays without the addition of a standard and measuring a signal for each assay;
e) calculating an average signal from said one or more additional assays without the addition of a standard and calculating a ratio of the average signal to a signal predicted by said first dose-response curve at a concentration of analyte of zero;
f) multiplying said first dose-response curve by said ratio to obtain a calibrated dose-response curve; and
g) employing said calibrated dose-response curve to obtain one or more inferred concentrations of the analyte from said one or more samples wherein the step a) of adding a known quantity of the analyte to one or more reagents provides a correlation between variations in the signal from an assay for which no additional standard or sample is added and variations in a signal from an assay for a standard having a finite quantity of the analyte.

20. The method according to claim 19 including repeating steps d) to g) for subsequent assays for one or more samples with unknown quantities of analyte.

21. The method according to claim 19 wherein the variation in the dose-response curve is caused by fluctuations or changes in temperature.

22. The method according to claim 19 wherein the variation in the dose-response curve is caused by fluctuations or changes in a quantity of aspirated reagents used in said assays.

23. The method according to claim 19 wherein the variation the dose-response curve is caused by fluctuations or changes in a quantity of dispensed reagents used in said assays.

24. A method of calibrating a dose-response curve of an assay for a target analyte, where the effects of both multiplicative scaling and translation variations in the dose-response curve, due to fluctuations in an assay parameter, on the determination of an unknown quantity of analyte in a sample are reduced or eliminated, comprising the steps of:
a) performing a set of initial assays for a plurality of standards with known analyte concentrations and measuring an assay signal for each standard;
b) fitting said measured signals and said known analyte concentrations to a predetermined mathematical function for generating a first dose-response curve;
c) modifying said first dose-response curve generated with said predetermined mathematical function by including a multiplicative scaling factor gₛ that multiplies all parameters in said predetermined mathematical function and a multiplicative translation factor gₜ that multiplies a concentration variable in said predetermined mathematical function, where said multiplicative scaling and translation parameters are unknown and represent possible variations in the first dose-response curve during future use of said assay, thereby obtaining a generalized dose-response curve;
d) performing assays for one or more samples with unknown quantities of analyte, together with performing assays for two additional standards, each standard having a different known concentration of analyte, where one or more assays is performed for each additional standard;
e) calculating an average signal from said assays of each of said additional standards, thereby obtaining two data pairs, each comprising a known concentration and a measured assay signal;
f) generating two equations using said two data pairs and said generalized dose-response curve and solving said two equations for values of said multiplicative scaling factor and said multiplicative translation factor;
g) generating a new dose-response curve by inserting said values of said multiplicative scaling factor and said multiplicative translation factor into said generalized dose-response curve; and
h) employing said new dose-response curve to obtain one or more inferred concentrations of said analyte from said one or more samples.

25. The method according to claim 24 including repeating steps d) to h) for subsequent assays for one or more samples with unknown quantities of analyte.

26. The method according to claim 24 wherein the variation in the dose-response curve is caused by fluctuations or changes in temperature.

27. The method according to claim 24 wherein the variation in the dose-response curve is caused by fluctuations or changes in a quantity of aspirated reagents used in said assays.

28. The method according to claim 24 wherein the variation the dose-response curve is caused by fluctuations or changes in a quantity of dispensed reagents used in said assays.

29. The method according to claim 24 wherein said dose response curve is a sigmoidal dose-response curve generally represented a mathematical function
$$S ( C ) = {a}_{2} + \frac{{a}_{1} - {a}_{2}}{1 + {( \frac{C}{{a}_{3}} )}^{a 4}} , \qquad ( 1 )$$
where S is the assay signal, C is the analyte concentration, and a₁-a₄ are parameters, and wherein a variation in an assay parameter that produces a multiplicative scaling and translation causes the dose-response curve to take a new form
$$S ( C ) = {g}_{s} [ {a}_{2} + \frac{{a}_{1} - {a}_{2}}{1 + {( \frac{{g}_{t} C}{{a}_{3}} )}^{a 4}} ] . \qquad ( 2 )$$
and wherein the new dose-response curve is obtained by the steps of, assaying two standards with concentrations C₁ and C₂ and recording their respective signals S₁ and S₂, and wherein the two data points S₁(C₁) and S₂(C₂) and equation (2) are then used to construct two equations with the two unknowns gₛ and gₜ, and wherein the solution of these equations provides gₛ and gₜ, enabling the accurate mathematical construction of the new dose-response curve shown in equation (2).
